(19) United States
(12) Patent Application Publication
(10) Pub. No.: US 2005/0076922 A1
(43) Pub. Date: Apr. 14, 2005

(54) Postural support therapy wall guard footrest and method

(76) Inventors: Scott W. Sears, Williston, VT (US); Holly Spence, South Burlington, VT (US)

Correspondence Address: Donald W. Meeker;Patent Agent, #E, 924 East Ocean Front, Newport Beach, CA, 92661, US

(21) Appl. No.: 10/683,367

(22) Filed: Oct. 9, 2003

(51) Int. Cl. A61F 5/37

(52) U.S. Cl. 128/882; 128/869

(57) ABSTRACT

A vertical sheet rests against a vertical surface with a bottom base support protrudes away from the device at a bottom edge resting on a horizontal surface supporting a user in a supine position with legs elevated, thighs vertical and calves horizontal. A vertical foot receiving area receives pressure from the bottom of the user's feet against the vertical surface and protects the vertical surface from contact by the feet. A horizontal heel support ledge, adjustably mounted on the vertical sheet, receives the heels to rest on and press down on the ledge.

### BACKGROUND OF THE INVENTION

1. Field of the Invention

The present invention relates to therapeutic devices and in particular to a therapeutic wall guard footrest and heel support used in postural support therapy exercising related to pelvic femoral positioning.

2. Description of the Prior Art

The body balances over the hips, which are two ball and socket joints. It is not uncommon for the body to become out of balance for a number of reasons including, but not limited to:

Stress, pain, genetic influences, handedness patterns, work habits, environmental influences, surgery, and respiratory patterns.

When we start to function out of balance, the body needs more muscle tone for function at work, play and rest. When one muscle becomes hypertonic (over-contracting) then compensatory muscle tension develops above, below, left and right of the original muscle tension. Over time there is an exponential increase in the demand on the muscles. As the muscle tension develops it creates increasing postural changes on the body, and creates a progressive increase in the pressure on the joints of the entire body. This creates an environment where the body is more vulnerable to injury, and the ability to heal from an injury is reduced. Most musculoskeletal ailments have some postural influences on them.

Clinically it has been documented that when the pelvis tips forward and or down there is accompanying femoral internal rotation. Increased tone in the psoas as well as the tensor fascia latea (TFL) have been sighted to be one cause of anterior pelvic tilting. When the femur is internally rotated secondary to this increased tone, the tensor fascia latea and gluteus minimus overcontract and add to femoral internal rotation. Significant correlations between hip medial rotation and hamstring flexibility have been found. The pathologic entity responsible for increased lateral patellofemoral force may be due to the underlying torsional direction of the femur's lumbopelvic position. Therefore, reducing an anterior tilted and forwardly rotated pelvis, through Protronics® resistance, reduces the demand on the posterior lengthened muscles (piriformis and hamstrings) and positionally shortened anterior muscles (psoas, TFL). The external and internal femoral rotators in conjunction with the repositioning hamstrings allow for stable pelvifemoral positional alignment.

Hip internal rotation and compensatory excessive tibial external rotation upon heel strike decreases the ability of the patella to translate laterally because of its conformity with the femoral trochlea and soft tissue attachments. An internally rotated femur and an unstable femur result in increased quadriceps tension and patellofemoral contact pressure. Protronics® resistance enables a more stable pelvifemoral relationship to be achieved. This results in decreased femoral internal rotation and increases the mechanical advantage of appropriate hip and knee stabilizers. In turn, there is decreased demand on lateral leg musculature (piriformis, vastus lateralis, biceps femoris, and tensor fascia latae) and an increased demand on appropriate synchronous quadriceps and hamstrings for dynamic postural support.

Hamstrings dynamically effect patellar stability by controlling tibial internal and external rotation. Balance of the medial and lateral static and dynamic stabilizers of the knee is necessary for proper pain-free function. Protronics® has been shown to increase hamstring EMG activity in normal subjects for certain activities. Applying Protronics® functional resistance and increasing proprioceptive input and output of the hamstrings reciprocally inhibits inappropriate firing of the psoas, vastus lateralis, and rectus femoris and facilitates appropriate feed-forward activation of both medial and lateral hamstrings.

Programmable torque (resistance) that is independent of speed and gravity applied to the hamstrings musculature permits isolated hamstring contractions independent of overused, neuronal patterned hip flexors ultimately repositioning the hip. Restoring the relationship of the femur to the patella during closed kinetic chain activity reduces the demand on lateral soft tissue (VL and IT band) enabling the patella to track appropriately, and in turn reduces lateral compression on the patellofemoral joint.

Protronics® has been shown to significantly improve function and reduce pain in subjects with patellofemoral pain.

In a prescribed exercise the patient lies in a supine position with feet up against a vertical surface, such as a wall. People are often reluctant to do the exercise at home for fear of marring the wall.

What is needed is a therapeutic wall guard footrest and heel support to be used in conjunction with postural support therapy related to pelvic femoral positioning to relieve muscle tension and pressure to restore posture.

### SUMMARY OF THE INVENTION

An object of the present invention is to provide a therapeutic wall guard footrest and heel support to be used in conjunction with postural support therapy related to pelvic femoral positioning to relieve muscle tension and pressure to restore posture.

A corollary object of the present invention is to provide a heel support positioned on a vertical surface above a reclined patient to enable the patient to apply both a forward pressure of the bottom of the patient's foot against the wall as well as a downward pressure of the patient's heels on the heel support to engage muscles not normally engaged in other ways, such as the hamstring and groin muscles.

A related corollary object of the present invention is to provide a wall guard footrest and heel support with a sufficiently large flat area to protect the wall from the feet of the user to prevent marring the wall.

Another object of the present invention is to provide a therapeutic wall guard footrest, which is adjustable and easy to use adaptable to patients of various heights with various leg lengths.

An additional object of the present invention is to provide a therapeutic wall guard footrest for use in postural support therapy, which has a sturdy vertical post that is attached to the bottom base support.

One more object of the present invention is to provide a therapeutic wall guard footrest of sheet plastic, which is durable and inexpensive to manufacture with a mass production molding process.

In brief, a therapeutic wall guard footrest that comprises a flat sheet, which has a vertical foot receiving area large enough to receive the feet of a user pressed flat against the vertical foot-receiving surface and prevent the feet from touching the wall. A height adjustable horizontal ledge heel support attached to the flat sheet provides a support for the heels of the user resting on and for downward heel pressure on the ledge to isolate and engage muscle groups not normally engaged. A bottom ridge protruding from the flat sheet away from the wall enables the flat sheet to be self-supporting against the wall.

The therapeutic wall guard is placed flat against the wall at a bottom end of a therapy table or on a floor or other horizontal surface with the bottom of the footrest resting on the horizontal surface. The patient lies face up on the horizontal surface with legs elevated in a position with the thighs vertical and the calves horizontal and the bottom of the feet pressing flat against the vertical surface and the heels resting and capable of pressing down on the height adjustable ledge of the therapeutic wall guard footrest. The therapeutic wall guard is used in conjunction with a variety of postural support therapy exercises related to pelvic femoral positioning wherein various muscles are stretched and relaxed with the legs up and the bottom of the feet against the wall and the heels pressing downwardly on the heel support horizontal ledge.

Postural Restoration is a physical therapy treatment approach based on the work of Ron Hruska MPA, P.T., and it is a neuromuscular retraining of the muscles in the body. This treatment isolates a contraction of the under-used muscles of the body and creates a corresponding inhibition of the opposing hypertonic muscles. Full recovery of length from the hypertonic muscles is expected, and this allows for full flexibility, full ROM, and an ability to recover proper posture/position. When good position is achieved the pressures within the tissues are minimized and the injured site is returned to the optimal position for healing.

The present invention gives the patient an anchor to help isolate the necessary muscles for adequate inhibition of the hypertonic muscles. Once neutral posture is achieved, further strengthening can be instituted to maintain proper muscle balance, and many of the strengthening exercises continue to use the present invention. Without the present invention patients always complain that they cannot reproduce the exercises effectively at home. Because all of the hypertonic muscles work in synergy, it is possible to create a relaxation of all the muscles of the lower and upper body when using the present invention. This list is far from inclusive, but it gives an idea of the scope of diagnoses that will benefit from using the present invention:
Foot and ankle: metatarsalgia, achilles tendonitis, plantar fascitis, and inversion sprains
Knee: patellofemoral syndrome, meniscus injuries, iliotibial band friction syndrome, Patella tendonitis, and ligament sprains
Hip: hip instability, greater trochanteric bursitis, piriformis syndrome, groin impingement, hip flexor strains, hamstring strains, and pelvic floor muscle dysfunction (incontinence)
Low back: generic low back pain, Sacroiliac joint pain, disc injuries, sciatica, spondylolisthesis, and stenosis
Thoracic: costochondritis and exercise induced asthma
Shoulder: rotator cuff tendonitis, glenohumeral joint impingement, subacromial bursitis, biceps tendonitis, and thoracic outlet syndrome
Cervical: neck pain, disc injuries, neck muscle tension, and anterior scalene syndrome
Temporal mandibular joint: muscle strains, cartilage wear and related headaches
Tension headaches
   The present invention can also be a contributing therapeutic factor in osteoarthritis of all joints.

An advantage of the present invention is in providing a device for use in postural support therapy related to pelvic femoral positioning.

Another advantage of the present invention is that it provides a wall guard that protects the wall from marks caused by a user's shoes or feet.

An additional advantage of the present invention is in providing a horizontal ledge heel support that may be vertically adjusted for individual users to rest the heels and apply downward pressure of the heels on the ledge.

A further advantage of the present invention is in providing a horizontal ledge that a user may rest their heels on in between exercises.

One more advantage of the present invention helps to significantly improve function and reduce pain in subjects with patellofemoral pain and all of the other problems listed above.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a front elevational view of the preferred embodiment of the therapeutic wall guard footrest positioned against a wall with the bottom resting on a horizontal surface;

FIG. 2 is a side elevational view with the wall and horizontal surface in partial section showing the preferred embodiment of the therapeutic wall guard footrest of FIG. 1 positioned against a wall with the bottom resting on a horizontal surface;

FIG. 3 is a perspective view of an alternate embodiment of the therapeutic wall guard footrest positioned against a wall with the bottom resting on a horizontal surface.

### BEST MODE FOR CARRYING OUT THE INVENTION

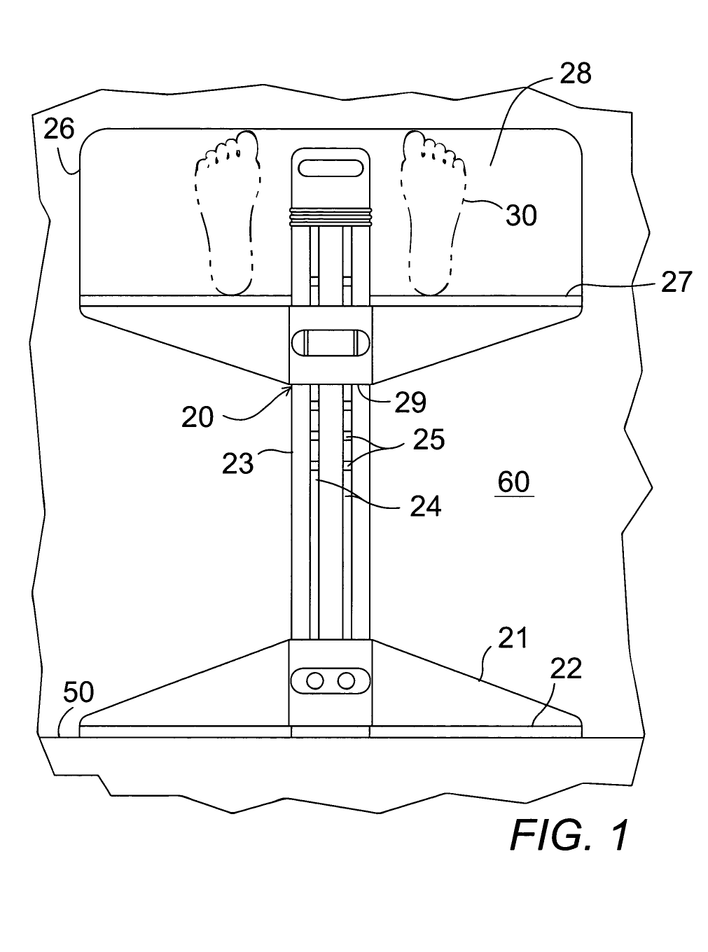

In FIGS. 1-3, a therapeutic wall guard footrest device 20 and 20A is used in postural support therapy related to pelvic femoral positioning and other applications with a patient in a supine position with the patient's feet 30 pressing against a vertical surface 60 with the patient's calves positioned horizontally.

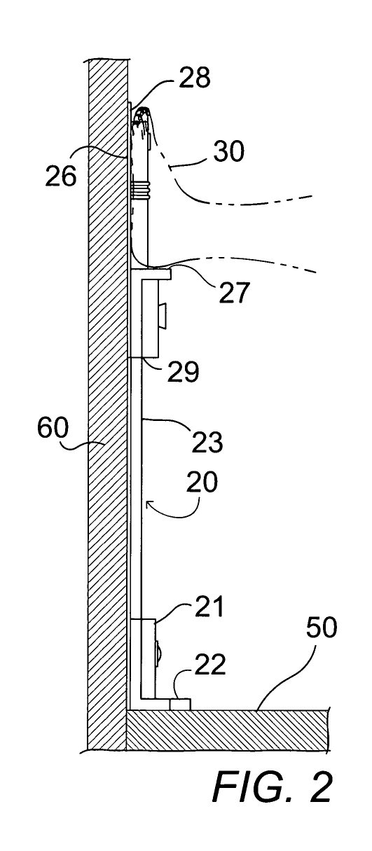

The device 20 and 20A comprises a vertical sheet 26 and 26A that has a foot receiving area 28 for the bottom of the feet sufficiently large to prevent the feet from touching the wall and a horizontal heel support ledge 27 and 27A which is movable vertically to accommodate users having different leg lengths. The device also comprises a bottom edge 22 and 22A, which protrudes away from the device at the bottom. The vertical sheet 26 and 26A is capable of resting against an external vertical surface 60, such as a wall, with the bottom edge 22 and 22A resting on an external horizontal surface 50, such as a padded table, bed or floor. The vertical sheet 26 and 26A is also capable of receiving the feet 30 of a user pressed against the foot receiving area 28 with the user's heels resting on and pressing down on the heel support ledge 27 and 27A, as shown in FIGS. 1 and 2. The foot receiving area 28 of the vertical sheet 26 prevents contact of the user's feet 30 with the external vertical surface 60. The horizontal heel support ledge 27 and 27A is capable of being adjusted so that a user who is supine on the horizontal surface 50 is able to rest his or her heels on the horizontal heel support ledge 27 and 27A with the user's feet flat against the foot receiving area 28 with the user's legs elevated so that his or her thighs are vertical and calves are horizontal. The vertical sheet 26 and 26A is capable of receiving pressure from the user's feet 30, which exert force against the vertical sheet 26 and 26A and the horizontal heel support ledge 27 and 27A is capable of supporting and receiving downward pressure from the heels of the user.

In FIGS. 1 and 2 the preferred embodiment of the therapeutic wall guard footrest device 20 comprises a vertical sheet 26 that has an integrated foot receiving area 28 and horizontal heel support ledge 27 and a vertical post 23. The vertical post 23 is attached to a bottom base support 21 with the bottom edge 22 protruding therefrom, as shown in FIGS. 1 and 2. The vertical sheet 26 further comprises a means for slidably engaging the vertical post 23 so that the integrated foot receiving area 28 and horizontal heel support ledge 27 are capable of moving vertically along the vertical post 23 and capable of being secured to the vertical post 23 at a desired height to accommodate a user's leg length. The means for slidably engaging the vertical post 23 comprises a sleeve 29 on the vertical sheet 26, which receives the vertical post 23 slidably within the sleeve 29. The vertical post 23 further comprises a vertical series of spaced horizontally paired openings 25 therethrough and a pair of threaded attaching means such as screws or bolts (not shown), which are capable of interconnecting the vertical sheet 26 to the vertical post 23 through any of the horizontally paired openings 25 to secure the horizontal heel support ledge 27 at a desired height. A pair of vertical grooves 24 serve as guides for mating protrusions (not shown) in the sleeve to assist in allowing the vertical sheet 26 to slide smoothly up and down the vertical post 23.

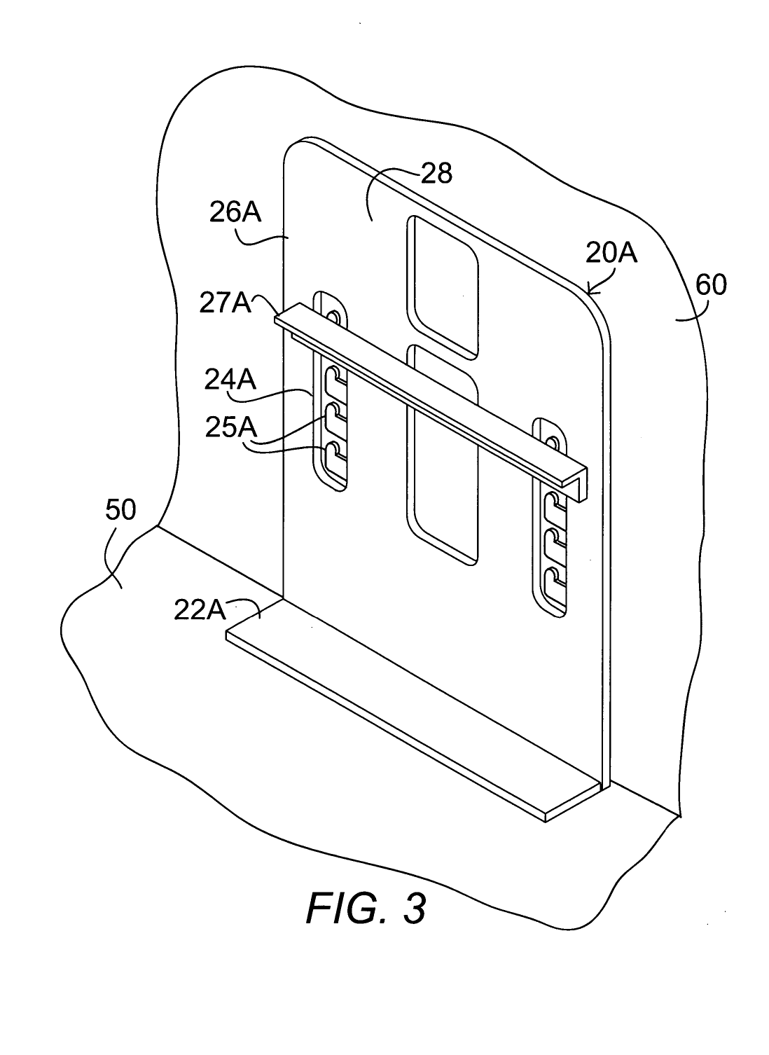

In FIG. 3, an alternate embodiment of the therapeutic wall guard footrest device 20A is shown positioned against a wall 60 with the bottom edge 22A resting on the horizontal surface 50. The vertical sheet 26A comprises at least one vertical slot 24A and an adjustable attaching means, preferably elongated threaded means such as screws or bolts (not shown), between the vertical sheet 26A and the horizontal heel support ledge 27A so that the horizontal heel support ledge 27A may be moved vertically relative to the vertical sheet 26A with the adjustable attaching means sliding in the vertical slot 24A. The adjustable attaching means is secured within the vertical slot 24A at various vertical locations, such as at one of the series of stops 25A in the vertical slot 24A, to accommodate users with various leg lengths with the stop engaging means contacting any one of the stops 25A.

In practice, a therapeutic method employs a wall guard footrest device 20 and 20A for use in postural support therapy related to pelvic femoral positioning and isolating and contracting specific muscle groups with a patient in a supine position and the patients feet 30 pressing against an external vertical surface 60.

The method comprises a first step of placing against an external vertical surface 60 a vertical sheet 26 or 26A that has a foot receiving area 28, a horizontal heel support ledge 27 or 27A mounted on the device by an adjustable means, and a bottom edge 22 or 22A protruding away from the device at the bottom. The vertical sheet 26 or 26A should be resting against the external vertical surface 60 with the bottom edge 22 or 22A resting on an external horizontal surface 50.

The method also comprises a second step of adjusting the horizontal heel support 27 or 27A so that the vertical sheet 26 or 26A is capable of receiving the feet 30 of a user pressed against the foot receiving area 28 with the user's heels resting on and pressing down on the heel support ledge 27 or 27A, as shown in FIGS. 1 and 2. The vertical sheet 26 or 26A prevents contact of the user's feet 30 with the external vertical surface 60. The horizontal heel support ledge 27 or 27A should be adjusted to a position on the device so that a user supine on the horizontal surface 50 is able to rest and press down his or her heels on the horizontal heel support ledge 27 or 27A, as shown in FIGS. 1 and 2, with the user's legs elevated so that his or her thighs are vertical and calves are horizontal. The foot receiving area 28 is capable of receiving pressure from the user's feet 30 when they exert a force against the vertical sheet 26 or 26A and the horizontal heel support ledge 27 and 27A is capable of supporting and receiving downward pressure from the heels of the user.

The user then rests on the horizontal surface 50, such as a padded table, bed, or floor with feet pressing flat against the foot receiving area 28 and heels resting on and pressing downwardly on the heel support ledge 27 and 27A and the user's legs elevated with calves horizontal and thighs vertical. In that position the user performs various therapeutic exercises in postural support therapy related to pelvic femoral positioning and isolating and contracting specific muscle groups.

A variety of exercise routines may be performed with the present invention including the following:

To perform the 90-90 Wall Hip Lift Exercise (starting position):

1. Lay on your back with your feet 30 flat on a wall 60, your knees and hips bent to 90°.
2. Place your hands on your lower ribs. Inhale through your nose, and then exhale through your mouth.
3. As you feel your ribs move down, raise your tailbone off the floor 50 by lifting your hamstrings. Keep your low back flat during the exercise.
4. Hold this position as you maintain a normal breathing pattern. Repeat the prescribed amount of times. Slowly lower your tailbone to the floor and rest 20 seconds.
5. Perform the prescribed amount of reps and sets.
   With hemibridge:
1. Assume the starting position.
2. Maintain hip lift with your left leg and straighten your right leg.
3. Lower and raise your straight right leg, on and off the wall 60 as you maintain a normal breathing pattern.
4. Perform the prescribed amount of reps and sets.
   With hip shift and ball (not shown):
1. Assume the starting position and place a 4-5″ or smaller ball between your knees.
2. As you maintain hip lift, shift your left hip down and your right hip up, so that you right knee is slightly above the left.
3. Take your bent right leg on and off the wall (the right thigh comes toward your chest).
4. Perform the prescribed amount of reps and sets.
   With medial hamstring:
1. Assume the starting position then place your left foot 30 and ankle slightly outward or internally rotate your left thigh while gently squeezing a ball.
2. Take your bent right leg on and off the wall 60 (right thigh comes towards your chest).
3. Perform the prescribed amount of reps and sets.
   With balloon (not shown):
1. Assume the starting position, raise your right arm above your head and hold a balloon in your left hand.
2. Inhale through your nose and slowly blow out into the balloon.
3. Pause 3 seconds with your tongue on the roof of your mouth to prevent airflow out of the balloon.
4. Without pinching the neck of the balloon, take another breath in through the nose. Slowly blow out again as you stabilize the balloon with your hand. Do not strain your neck or cheeks as you blow.
5. After the fourth breath in, pinch the balloon neck and remove it from your mouth and let the air out.
6. Perform the prescribed amount of reps and sets.

It is understood that the preceding description is given merely by way of illustration and not in limitation of the invention and that various modifications may be made thereto without departing from the spirit of the invention as claimed.

1. A therapeutic wall guard footrest device for use in postural support therapy related to pelvic femoral positioning and isolating and contracting specific muscle groups with a patient in a supine position having the patients feet pressing against a vertical surface, the device comprising:
a vertical sheet comprising a foot receiving area sufficiently large to receive the bottom of the feet of a user and prevent the feet from touching an external surface behind the foot receiving area, a horizontal heel support ledge mounted on the vertical sheet by an adjustable means, and a bottom base support protruding away from the vertical sheet at a bottom edge of the device, the vertical sheet capable of resting against an external vertical surface with the bottom edge and base support resting on an external horizontal surface, the vertical sheet capable of receiving the feet of a user pressed against the foot receiving area with the user's heels resting on the heel support ledge, the foot receiving area preventing contact of the user's feet with the external vertical surface, and the horizontal heel support ledge capable of being adjusted to a position on the vertical sheet so that a user supine on the horizontal surface is able to rest the user's heels on and apply downward pressure by the heels on the horizontal heel support ledge with the user's legs elevated so that the user's thighs are vertical and the user's calves are horizontal with the user's feet flat against the foot receiving area, the vertical sheet being capable of receiving pressure from the user with the bottom of the user's feet exerting a force against the vertical sheet and the horizontal heel support ledge is capable of supporting and receiving downward pressure from the heels of the user.

2. The device of claim 1 wherein the vertical sheet comprises an integrated foot receiving area and horizontal heel support ledge and further comprising a vertical post attached to the bottom base support and the vertical sheet further comprises a means for slidably engaging the vertical post so that the integrated foot receiving area and horizontal heel support ledge is capable of moving vertically along the vertical post and capable of being secured to the vertical post at a desired height to accommodate a user's leg length.

3. The device of claim 2 wherein the means for slidably engaging the vertical post comprises a sleeve on the vertical sheet capable of receiving the vertical post slidably within the sleeve.

4. The device of claim 3 wherein the vertical post further comprises a vertical series of spaced horizontally paired openings therethrough and a pair of threaded attaching means are capable of interconnecting the vertical sheet to the vertical post through any of the horizontally paired openings to secure the horizontal heel support ledge at a desired height.

5. The device of claim 1 wherein the vertical sheet further comprises at least one vertical slot and further comprising an adjustable attaching means between the vertical sheet and the horizontal heel support ledge so that the horizontal heel support ledge is capable of moving vertically relative to the vertical sheet with the adjustable attaching means sliding in the at least one vertical slot and the adjustable attaching means is capable of being secured within the at least one vertical slot at various vertical locations to accommodate users with various leg lengths.

6. The device of claim 5 wherein the attaching means comprises an elongated threaded means attachable between the vertical sheet and the horizontal heel support ledge.

7. The device of claim 5 wherein the vertical slot is provided with a series of stops and the attaching means comprises a stop engaging means securable to the horizontal heel support ledge, the stop engaging means capable of contacting any one of the stops.

8. A therapeutic method employing a wall guard footrest device for use in postural support therapy related to pelvic femoral positioning and for isolating and contracting specific muscle groups with a patient in a supine position having the patients feet pressing against a vertical surface, the method comprising:
a first step of placing against an external vertical surface a vertical sheet comprising a foot receiving area, a horizontal heel support ledge mounted on the vertical sheet by an adjustable means, and a bottom base support protruding away from the device at a bottom edge of the device, the vertical sheet resting against the external vertical surface with the bottom edge and base support resting on an external horizontal surface;
a second step of adjusting the horizontal heel support ledge so that the vertical sheet is capable of receiving the feet of a user pressed against the foot receiving area with the user's heels resting on and pressing down on the heel support ledge, the foot receiving area preventing contact of the user's feet with the external vertical surface, and the horizontal heel support ledge adjusted to a position on the vertical sheet so that a user supine on the horizontal surface is able to rest the user's heels on and press the heels down on the horizontal heel support ledge with the user's legs elevated so that the user's thighs are vertical and the user's calves are horizontal with the user's feet flat against the foot receiving area, the vertical sheet being capable of receiving pressure from the user exerting a force against the vertical sheet and the horizontal heel support ledge being capable of supporting and receiving downward pressure from the heels of the user.

9. The method of claim 8 wherein the vertical sheet comprises an integrated foot receiving area and horizontal heel support ledge and further comprising a vertical post attached to the bottom base support and the vertical sheet further comprises a means for slidably engaging the vertical post so that the integrated foot receiving area and horizontal heel support ledge is capable of moving vertically along the vertical post and capable of being secured to the vertical post at a desired height to accommodate a user's leg length.

10. The method of claim 9 wherein the means for slidably engaging the vertical post comprises a sleeve on the vertical sheet capable of receiving the vertical post slidably within the sleeve.

11. The method of claim 10 wherein the vertical post further comprises a vertical series of spaced horizontally paired openings therethrough and a pair of threaded attaching means are capable of interconnecting the vertical sheet to the vertical post through any of the horizontally paired openings to secure the horizontal heel support ledge at a desired height.

12. The method of claim 8 wherein the vertical sheet further comprises at least one vertical slot and further comprising an adjustable attaching means between the vertical sheet and the horizontal heel support ledge so that the horizontal heel support ledge is capable of moving vertically relative to the vertical sheet with the adjustable attaching means sliding in the at least one vertical slot and the adjustable attaching means is capable of being secured within the at least one vertical slot at various vertical locations to accommodate users with various leg lengths.

13. The method of claim 12 wherein the attaching means comprises an elongated threaded means attachable between the vertical sheet and the horizontal heel support ledge.

14. The method of claim 12 wherein the vertical slot is provided with a series of stops and the attaching means comprises a stop engaging means securable to the horizontal heel support ledge, the stop engaging means capable of contacting any one of the stops.
